(19) United States
(12) Patent Application Publication
(10) Pub. No.: US 2007/0249024 A1
(43) Pub. Date: Oct. 25, 2007

(54) Novel Dna Synthesis Technology with 3'-Beaded Oligo Dna and Dna Polymerase

(75) Inventor: Kanae Muraiso, Rockville, MD (US)

Correspondence Address: OMORI & YAGUCHI USA, LLC, EIGHT PENN CENTER, SUITE 1901, 1628 JOHN F. KENNEDY BOULEVARD, PHILADELPHIA, PA, 19103, US

(73) Assignees: KANAE MURISO, ROCKVILLE, MD 20852-6677, MD (US); BISO-INFO-DESIGN, INC., ROCKVILLE MD,, MD (US)

(21) Appl. No.: 11/628,562

(22) PCT Filed: Jun. 3, 2005
(86) PCT No.: PCT/US05/19744

§ 371 Date: Dec. 4, 2006

Related U.S. Application Data
(60) Provisional application No. 60/576,728, filed on Jun. 3, 2004.

(51) Int. Cl. C12P 19/34; C12M 1/00

(52) U.S. Cl. 435/91.5; 435/283.1

(57) ABSTRACT

A method of synthesizing a desired DNA having a predetermined sequence, comprising the steps of (a) preparing, based on the desired DNA, a plurality of 3′-biotin-immobilized oligo DNA having a fixed length; (b) preparing a starting DNA which has a complementary sequence to a 3′ end of a first immobilized oligo DNA of the plurality of oligo DNA, and wherein the starting DNA has a length shorter than the length of the first immobilized oligo DNA; (c) annealing the starting DNA with the first immobilized oligo DNA, extending the starting DNA to complement the first immobilized oligo DNA, thereby making a newly extended DNA; (d) denaturing a first double strand DNA consisting of the newly extended DNA and the first immobilized oligo DNA; (e) collecting the extended DNA by removing the first immobilized oligo DNA from the extended DNA; (f) annealing the collected, extended DNA with a second immobilized oligo DNA from the plurality of oligo DNA, further extending the extended DNA to complement the second oligo DNA, thereby making a further extended DNA; (g) denaturing a second double stranded DNA consisting of the second immobilized oligo DNA and the further extended DNA; (h) collecting the further extended DNA by removing the second immobilized oligo DNA from the extended DNA; and (i) repeating steps (f) through (h) until the further extended DNA becomes the predetermined sequence thereby completing synthesis of the desired DNA.

### PRIORITY FILING

This application claims priority from co-pending Provisional Patent Application Ser. No. 60/576,728 entitled “A Novel DNA Synthesis Technology With 3′-Beaded Oligo DNA and DNA Polymerase”, filed on Jun. 3, 2004.

### FIELD OF THE INVENTION

The present invention relates generally to DNA synthesis and more particularly, to a unique, simple and cost-effective Deoxyribonucleic acid (DNA) polymerase-based DNA synthesis technology.

### BACKGROUND OF THE INVENTION

DNA is an essential and necessary molecule in life science research, from the basic to the clinical fields. In basic biomedical research laboratories, DNA is widely used as a central player in many applications such as Polymerase Chain Reaction (PCR), gene (protein) expression, structural biology, the synthesis of dominant negative mutants, functional transgenic mice, gene knockout mouse studies, and the synthesis of antigens for vaccine development. Furthermore, the use of recombinant DNA is already inevitable for clinical fields. It is widely known that several growth factors, interferon, and insulin are commonly used for specific therapeutic purposes. All of such therapeutic molecules were created from recombinant DNA.

Since the international genome project completed the entire sequencing of all ˜30,000 genes of human genome, it is now possible to work with them and study the functioning of each gene, and explore more therapeutic targets for different diseases. Performing such studies requires methods of useful and easy-access recombinant DNA synthesis. It follows that the life science and biomedical markets in which DNA synthesis can be introduced continues to expand.

In this regard, single and double strand DNA syntheses should be discussed separately. To date, many commercial bio-companies such as the Invitrogen Corporation (www.invitrogen.com), Qiagen, Inc. (www.qiagen.com), and the Sigma-Aldrich Corporation (www.sigma-aldrich.com) have services for custom DNA syntheses. However, they have length limitations: single strand DNA can only be prepared up to 50-100 nucleotides in length.

Regarding double strand DNA, there currently exists no common synthetic technology to create long double strand DNA for protein research or gene knockout studies. Thus, researchers typically must amplify their target/interest DNA by PCR technology using oligo DNA. Some companies do offer synthetic DNA services, such as “GeneMaker™” of Blue Heron Biotechnology, Inc. (www.blueheronbio.com). They offer DNA synthesis for any length. However, such synthesis is extremely expensive (at greater than $3.50/base pair). Thus, it is not realistic to synthesize any double strand DNA using such expensive methodologies.

Accordingly, a method for producing double strand DNA cost effectively, without limitations on the final length is highly desired.

### SUMMARY OF THE INVENTION

A method of synthesizing a desired DNA having a predetermined sequence, characterized by the steps of (a) preparing, based on the desired DNA, a plurality of 3′-biotin-immobilized oligo DNA having a fixed length; (b) preparing a starting DNA which has a complementary sequence to a 3′ end of a first immobilized oligo DNA of the plurality of oligo DNA, and wherein the starting DNA has a length shorter than the length of the first immobilized oligo DNA; (c) annealing the starting DNA with the first immobilized oligo DNA, extending the starting DNA to complement the first immobilized oligo DNA, thereby making a newly extended DNA; (d) denaturing a first double strand DNA consisting of the newly extended DNA and the first immobilized oligo DNA; (e) collecting the extended DNA by removing the first immobilized oligo DNA from the extended DNA; (f) annealing the collected, extended DNA with a second immobilized oligo DNA from the plurality of oligo DNA, further extending the extended DNA to complement the second oligo DNA, thereby making a further extended DNA; (g) denaturing a second double stranded DNA consisting of the second immobilized oligo DNA and the further extended DNA; (h) collecting the further extended DNA by removing the second immobilized oligo DNA from the extended DNA; and (i) repeating steps (f) through (h) until the further extended DNA becomes the predetermined sequence thereby completing synthesis of the desired DNA.

It is embodied in another mode of the invention an apparatus for synthesizing a desired DNA having a predetermined sequence, which uses, characterized by (a) a first device for annealing a starting DNA with a first oligo DNA of a plurality of oligo DNA, where the starting DNA has a complementary sequence to a 3′ end of the first oligo DNA, and where the plurality of oligo DNA have a fixed length and the starting DNA has a length shorter than the length of the oligo DNA, thereby extending the starting DNA to complement the first oligo DNA and making a newly extended DNA; (b) a second device for denaturing to remove the first oligo DNA from the extended DNA and collect the extended DNA; (c) a third device for transferring the collected, extended DNA to a next oligo DNA from the plurality of oligo DNA; (d) a fourth device for annealing the extended DNA with the next oligo DNA, further extending the extended oligo DNA to complement the next oligo DNA, thereby making a further extended DNA; (e) a fifth device for denaturing to remove the next oligo DNA from the further extended DNA and collect the further extended DNA; (f) a sixth device for transferring the collected, extended DNA to a next oligo DNA; and (g) a seventh device for repeating steps (d) through (f) until the further extended DNA becomes the predetermined sequence thereby completing the desired DNA.

### BRIEF DESCRIPTION OF THE FIGURES

FIG. 1 shows the 3′-beaded oligo DNA #1 and starting DNA of the present invention;

FIG. 2 shows the annealing step of the present invention;

FIG. 3 shows the extension step of the present invention;

FIG. 4 further illustrates the extension step, showing how the starting DNA has been extended to complement the 3′-beaded oligo DNA #1;

FIG. 5 shows the denaturing step of the present invention;

FIG. 6 shows the removal of the 3′-beaded oligo DNA #1 by means of a magnet;

FIG. 7 shows the 3′-beaded oligo DNA #2 and the extended DNA of the present invention;

FIG. 8 shows the second annealing step;

FIG. 9 shows the second extension step;

FIG. 10 further illustrates the second extension step, showing how the extended DNA has been extended to complement the 3′-beaded oligo DNA #2;

FIG. 11 shows the removal of the 3′-beaded oligo DNA #2 by means of a magnet;

FIG. 12 shows the 3′-beaded oligo DNA #3 and the extended DNA of the present invention;

FIGS. 13-17 illustrate the repetition of the steps shown in the above FIGS. 8-11 using oligo #3 in place of oligo #2;

FIGS. 18A and B show the extension of 20 mer DNA of T7 primer (starting DNA) to 67 mer DNA (extended DNA), using the method embodied in the present invention;

FIG. 19 shows the PCR amplification of the final product to verify its length using T7 and SP6 primers;

FIGS. 20A and B show the extension of 20 mer DNA of T7 primer to 241 mer DNA, using the method embodied in the present invention;

FIGS. 21 and 22 are summarized overviews of the present invention relating to DNA synthesis; and

FIG. 23 shows an applied DNA synthesis technology of the present invention using a device having immobilized oligo DNA.

### DETAILED DESCRIPTION OF THE INVENTION

A method of DNA synthesis, based on 3′-beaded oligo DNA and in accordance with an aspect of the present invention, will be described with reference to the above-described figures as follows. As a method based on Taq-DNA polymerase and cycled extensions, similar to PCR, it is a particularly cost effective technique as compared with producing synthetic DNA.

Essentially, this method may involve the use of the following reagents, materials and equipment, as commonly known in the art:

(1) 3′-biotin modified oligonucleotide DNA (˜47-49 nucleotides, or mer, for example);

(2) starting DNA (of >or =17 mer);

(3) beads for biomagnetic separation, (Dynabeads M-280 from Dynal Biotech are used in this example);

(4) a magnet;

(5) 10×PCR buffer;

(6) Taq polymerase;

(7) dNTP;

(8) ddH₂O; and

(9) a thermal cycler.

The method involves first preparing the oligonucleotide DNA, which are 3′-terminal oligonucleotides modified with biotin #1. It should be noted that, at first, the 3′-biotin-modified oligo DNA do not have the above-listed magnetic beads bound to them. They become bound to the streptavidin-coupled beads through following the Dynal protocol used in this example.

The 3′-biotin modified DNA are immobilized to the Dynabeads™ M-280 by following the protocol of Dynal Biotech, thereby producing 3′-beaded oligonucleotides.

A starting DNA has been prepared, having at least a 17 mer complementary sequence to a first 3′-beaded oligo (oligo DNA #1). Then, these oligo DNA #1 and starting DNA are mixed in a PCR tube in the following concentrations:

| | | |
|---|---|---|
| (1) 10× PCR buffer (with MgCl₂) | 5 | ul |
| (2) starting DNA (10 uM) | 1 | ul |
| (3) 3′-beaded oligo DNA #1 (1 uM) | 0.1 | ul |
| (4) dNTP (10 uM) | 1 | ul |
| (5) Taq polymerase (2.5 U/ul) | 0.125 | ul |
| (6) ddH₂O | 42.775 | ul |
| Total | 50 | ul |

The tube is then placed in the thermalcycler, and the following protocol is followed:

(1) start with 94° C. for 3 minutes;

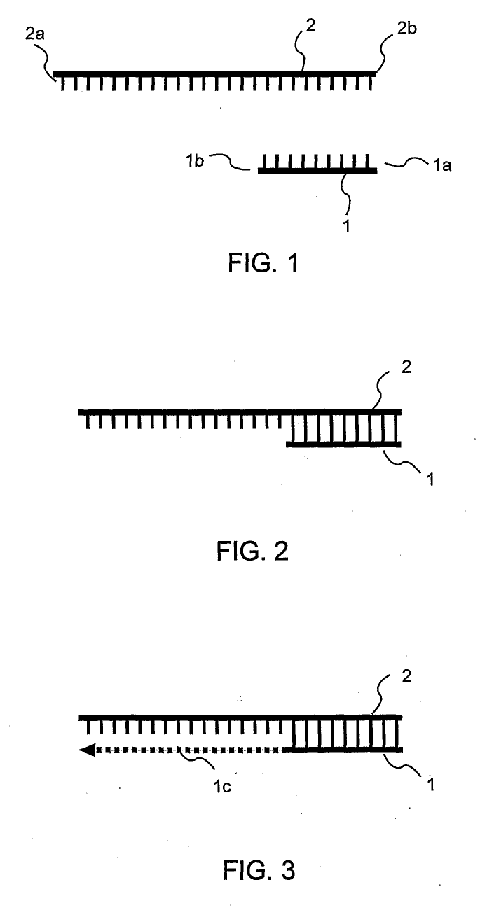

(2) 94° C. for another 30 seconds, for denaturing into single strand DNA, such as starting DNA, indicated as 1, and the complementary 3′-beaded oligo DNA #1, indicated as 2; in FIG. 1. The 5′ and the 3′ ends of the oligo #1 are indicated as 2a and 2b, respectively, and the 5′ and the 3′ ends of the starting DNA are indicated as 1a and 1b, respectively.

(3) 58° C. for 30 seconds for the annealing step of this aspect of the present invention, as illustrated in FIG. 2, where the oligo #1 and the starting DNA bind at their complementary sequences;

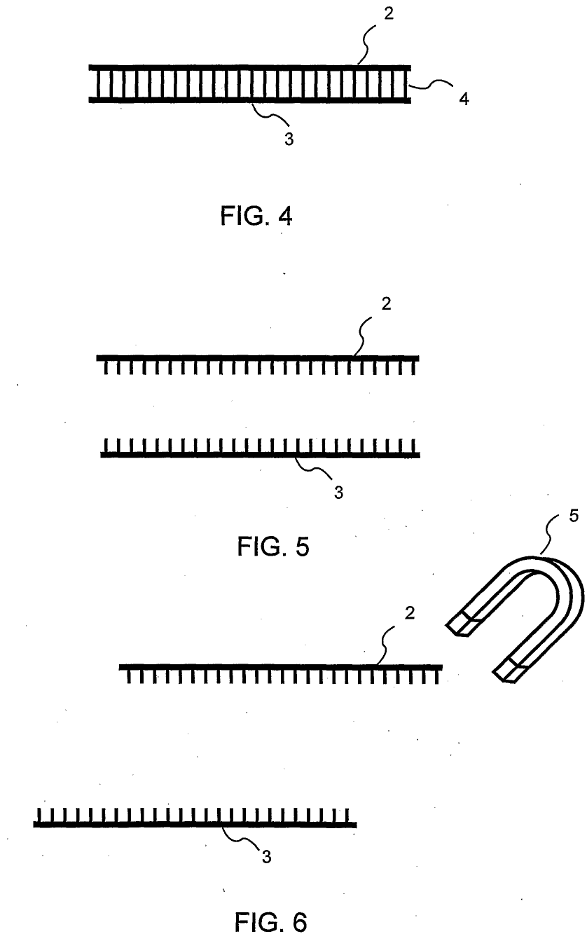

(4) 72° C. for 30 seconds for the extension step, as referenced by 1c in FIG. 3. Through DNA synthesis from the Taq polymerase, the starting DNA 1 is extended to be complementary to the oligo #1, as shown at 1c. The resulting extended DNA strand is shown as 3 in FIG. 4, where the oligo #1 2 and the extended DNA 3 make up a double strand DNA 4;

(5) the PCR tubes are then mixed vigorously, by a means known to those of ordinary skill in the art;

(6) the above protocol steps (2) through (5) are repeated for 10 cycles;

(7) after the 10 cycles of PCR reactions, the DNA is then denatured using a 95° C. heat block for 5 minutes, as illustrated in FIG. 5, where the oligo #1 2 strand and the extended DNA strand 3 are separated;

(8) the mixture is immediately transferred to the magnet 5 and incubated for 30 seconds to 1 minute, where the bead-bound oligo #1 2 is drawn to the magnet 5 side for recovery, as illustrated in FIG. 6;

(9) the supernatant is then transferred to a new PCR tube, such as through aspiration, thereby collecting the beaded oligo #1, leaving only the newly extended DNA 3;

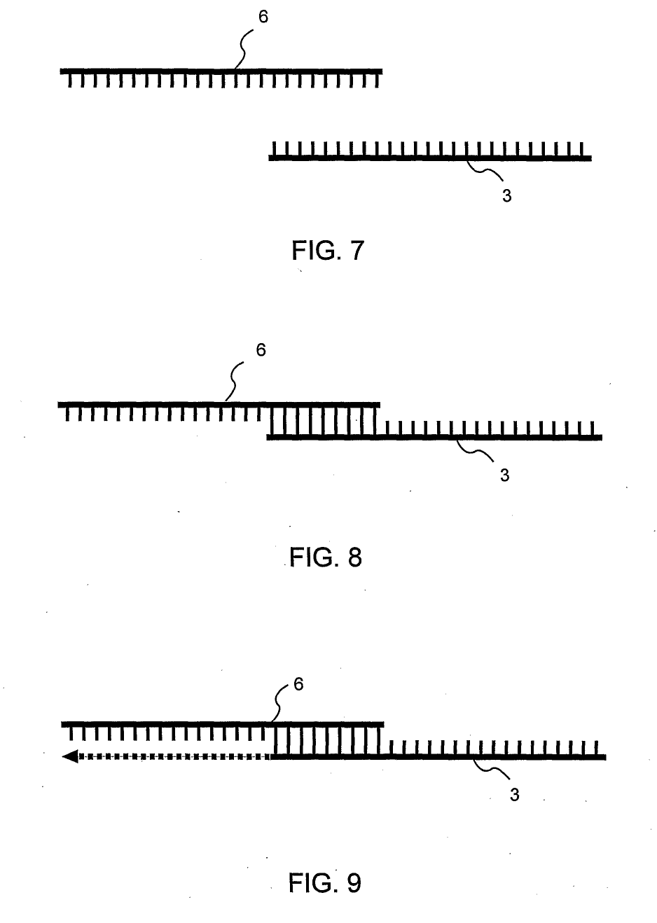

(10) the next 3′-beaded oligo #2 6 is then added, as shown in FIG. 7; and

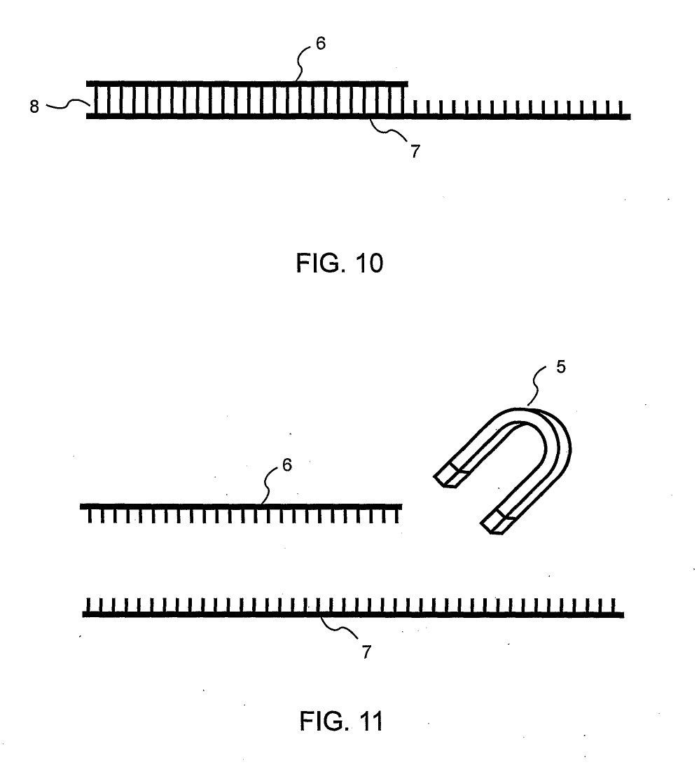

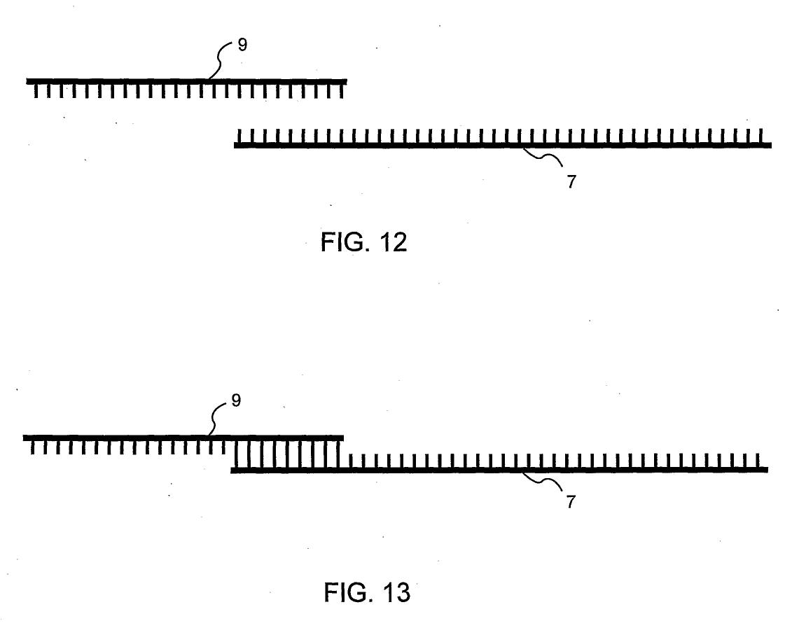

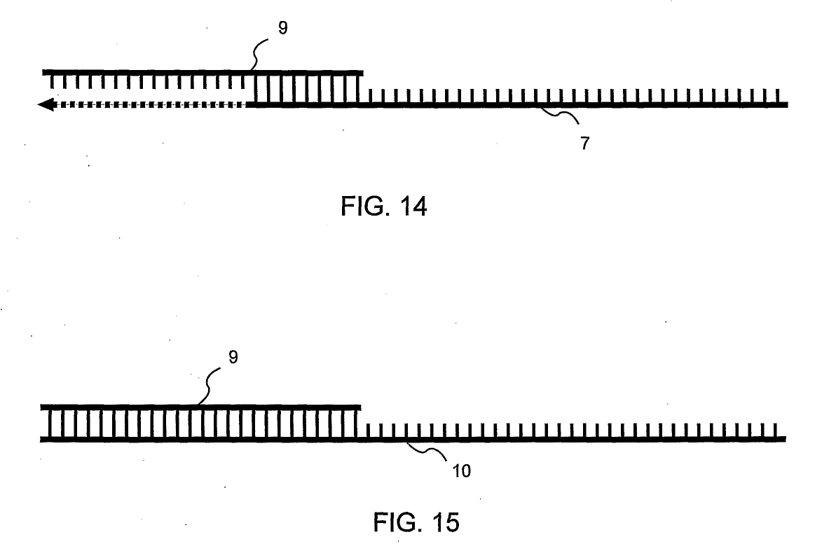

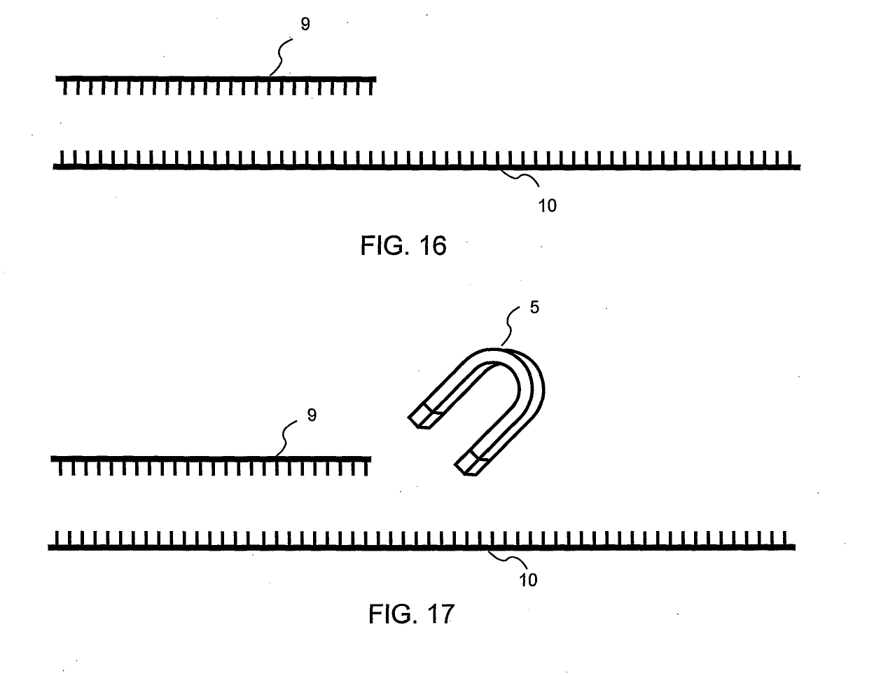

(11) the above steps (1) through (10) are then repeated, thereby successively elongating the starting DNA. FIGS. 7-11 show the repetition of the above steps using the oligo #2 6, where FIGS. 9 and 10 illustrate how the extended DNA 3 elongates further, to complement the oligo #2 6, due to the DNA synthesis. This results in a further extended DNA 7 and another double strand DNA 8. FIGS. 12-17 illustrate the further repetition of these steps, using the next oligo #3 9, resulting in a still further extended DNA 10.

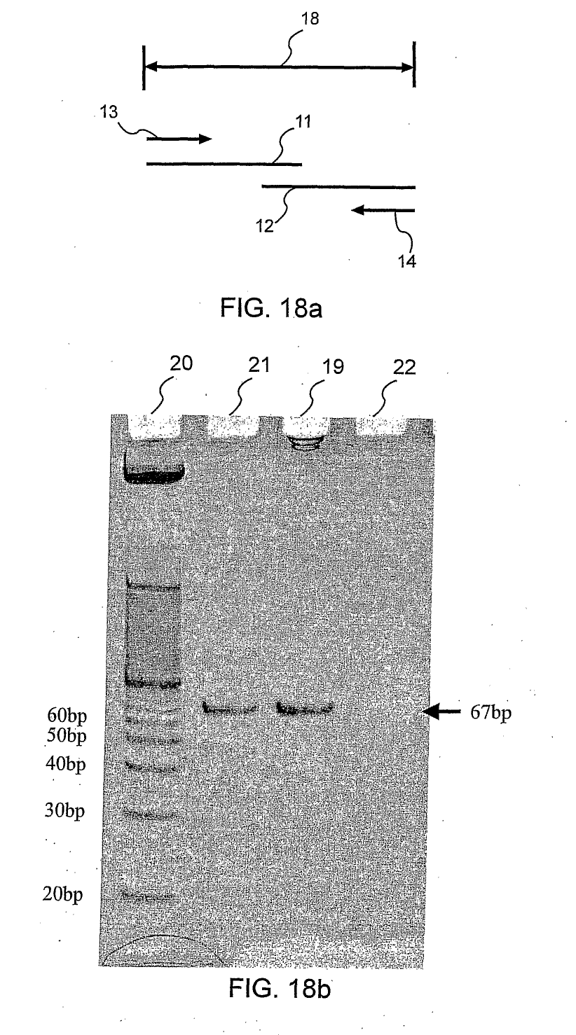

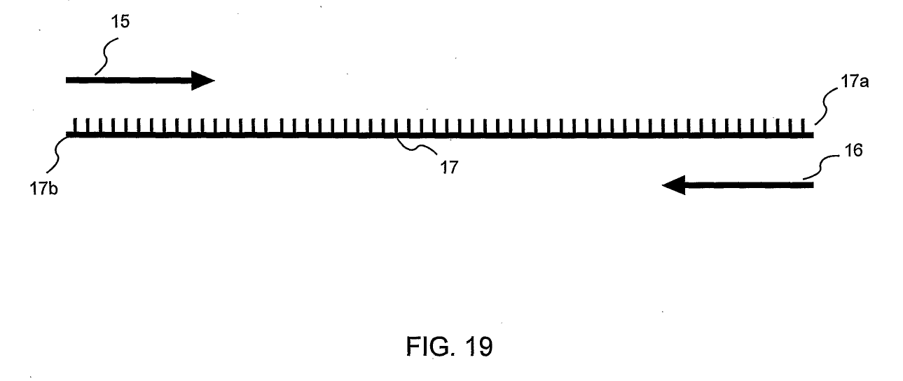

In our experiments using the above method, we first attempted to synthesize a 67 nucleotide length (mer) of DNA, as a demonstration of short DNA synthesis. As shown in FIG. 18A, two different (47 mer 11 and 48 mer 12) 3′-beaded oligo DNA were used. The starting DNA was a T7 primer (20 mer) 13 and the closing sequence corresponded to the SP6 primer 14 sequence. The sequence of the final product was verified by PCR using T7 15 and SP6 16 primers to bracket the target region to be verified, as shown in FIG. 19, where the final product 17 has the 5′ end at 17a and 3′ end at 17b.

As shown in FIG. 18A, the 20 mer starting DNA 13 which is a T7 primer, was successfully extended to the targeted length of 67 base-pairs (bp), as indicated as 18. FIG. 18B shows the photograph of the results visualized on a 7.5% polyacrylamide gel. As shown the extended sample 19 has 67 bp in comparison with the marker 20, as does the positive control 21, whereas the negative control 22 shows no results.

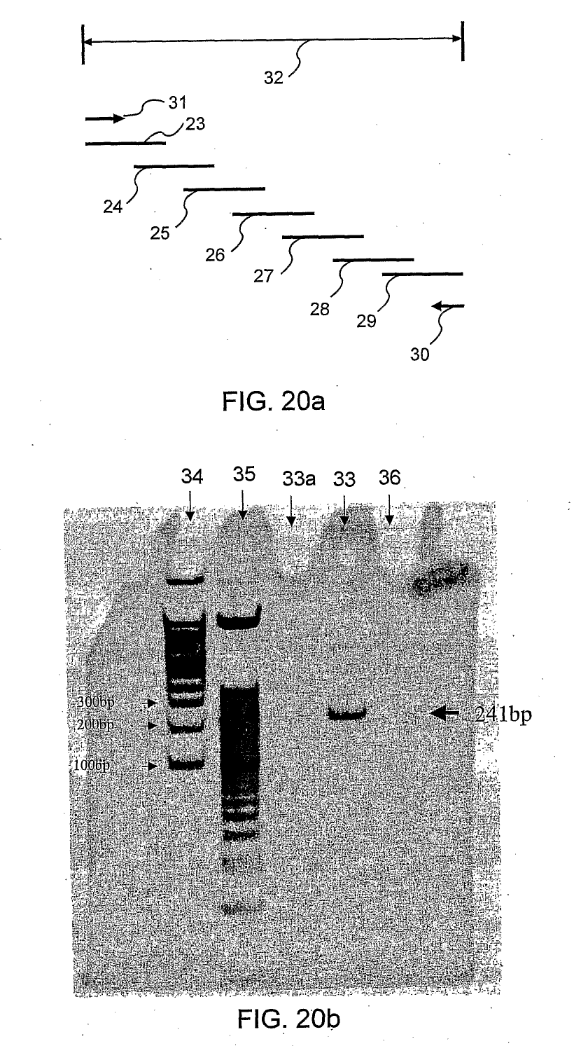

Our second attempt was to synthesize a long, 241 nucleotide length of DNA. As illustrated in FIG. 20A, seven different and sequential 49 mer 3′-beaded oligo DNA 23-29 were prepared. SP6 (24 mer) 30 was used as the starting DNA, and the closing sequence corresponded to the T7 (20 mer) 31 sequence. A successful extension to the 241 bp length was achieved, as indicated as 32.

FIG. 20B shows the photograph of the results, as visualized on a 7.5% polyacrylamide gel. As shown a 241 bp length of DNA corresponding to a part of a luciferase gene, was successfully synthesized. This successful final product samples is indicated as 33, in comparison with the 100 bp 34 and 10 bp 35 markers. The negative control result is shown as 36.

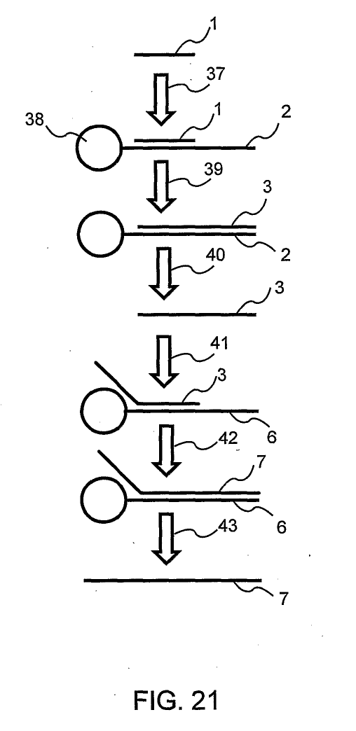

FIG. 21 illustrates the above described cycles. At step 37, the starting DNA 1 binds at the complementary sequence to the oligo #1 2, which have been immobilized to beads 38. At step 39, the starting DNA 1 is extended to complement the beaded oligo #1 2, then denatured at step 41 to be isolated from the oligo #1. This cycle gets repeated at step 4 1, where the newly extended DNA 3 is annealed to the beaded oligo #2 6 at their complementary sequence, then further extended at step 42. Another denaturing and isolation of the further extended DNA 7 occurs at step 43.

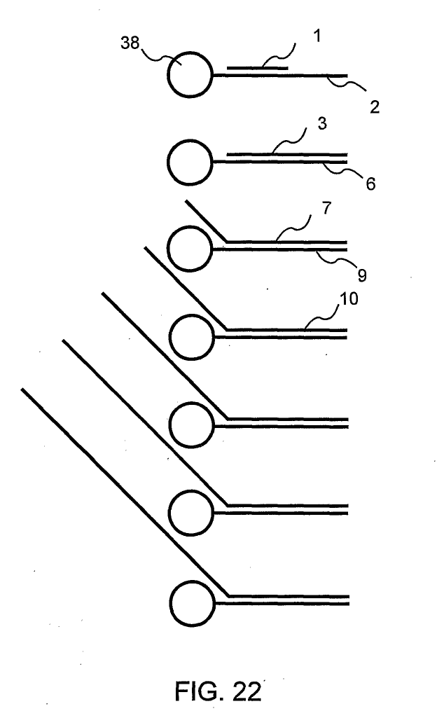

FIG. 22 illustrates the repeated, sequential extensions of the starting DNA 1 and extended DNA, at each cycle using the respective 3′-beaded oligo DNA, until the extended DNA becomes the predetermined, desired sequence.

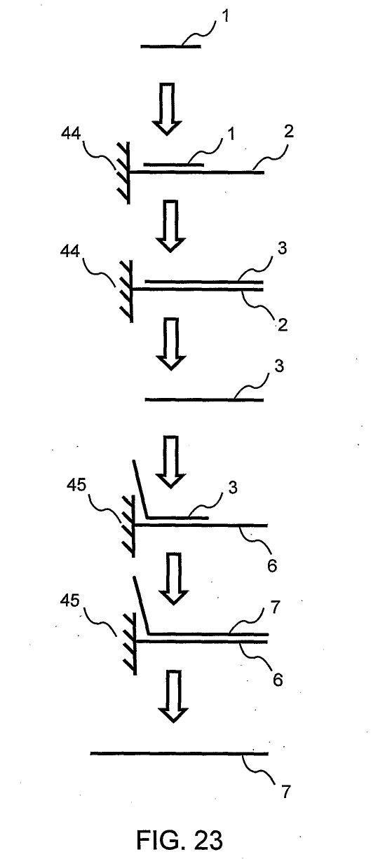

FIG. 23 depicts a variation on the above mode, where the oligonucleotide DNA is synthesized on a custom DNA chip and immobilized on this DNA microarray device. Thus, rather than utilizing the beads as in FIGS. 22 and 23, the oligo DNA are spotted, such as oligo #1 at Spot 1 44, oligo #2 at Spot 2 45, etc. The above-described cycle of annealing, extension, and denaturing can then be performed and repeated at its respective spot, for large-scale DNA synthesis.

It should be noted that, after each 10 cycles of denaturing, annealing, and extension, the mixture in the PCR tube should be mixed vigorously, as described at step (5) above. This technical point is necessary to prevent the precipitation of the beaded oligo at the bottom of the tube. A comparison of the 3-6-04 33a and 3-8-04 33 results in FIG. 20B demonstrates this. The 3-6-04 result 33a, showing unsuccessful reactions, corresponds to a non-mixed sample. The 3-8-04 result 33, indicating successful extension to 241 bp, corresponds to a vigorously mixed sample.

As noted above, it is also important to have at least 17 mer of corresponding sequence between the 3′-beaded oligo and the starting/extended DNA, that can anneal to each other.

A limitation of this method may be that the efficiency of annealing and extension was under 100% in the experiments. Also, if the target DNA has highly repetitive sequences, this may cause problems. The extending DNA may anneal among these repetitive sequences, thereby interfering with the new extensions.

As stated above, because of this method's similarity to PCR and its basis on Taq-DNA polymerase and cycled extensions, it has significant cost effectiveness when compared with the production of synthetic DNA. This method may provide significant cost reductions compared to conventional phosphoramadite methods used by the existing biotechnology companies such as Qiagen, Inc., the Sigma-Aldrich Corporation, and others. This is because such DNA synthesis can encounter technical difficulties synthesizing DNA at longer lengths, such as the above-described 241 nucleotide length.

The previously-mentioned Blue Heron Biotechnology, Inc. currently offers DNA synthesis at any length. However, at greater than $3.50/base pair, their gene synthesis platform is very expensive. In comparison with such biotech companies, our technology provides a lower cost of under $1.00/base pair.

The present invention also provides the synthesis of desired DNA without limitation in terms of the final length. This offers a highly advantageous feature, when compared with the conventional technologies of long DNA synthesis. The present invention involves the above-described 17 mer annealing along with simple, cycled extensions. The manufacturing of long synthesis DNA using conventional techniques, on the other hand, typically encounters technical difficulties.

The foregoing description of the embodiments of this invention has been presented for purposes of illustration and description. It is not intended to be exhaustive or to limit the embodiments of the invention to the form disclosed, and, obviously, many modifications and variations are possible. As an example, other methods and devices for carrying out the above cycles of annealing, extension, and denaturing using the various oligo DNA, as commonly known in the art, may also be utilized. Future enhancements to DNA and oligonucleotide microarray technologies may further improve this method's cost effectiveness and speed in DNA synthesis. Such modifications and variations that may be apparent to a person skilled in the art are intended to be included within the scope of this invention as defined by the accompanying claims.

### EXPLANATION OF REFERENCE SIGNS

1 starting DNA
1a 5′ end of starting DNA
1b 3′ end of starting DNA
1c extension of starting DNA
2 first immobilized oligo DNA
2a 5′ end of first immobilized oligo DNA
2b 3′ end of first immobilized oligo DNA
3 extended DNA
4 first double strand DNA
5 magnet
6 second immobilized oligo DNA
7 further extended DNA
8 second double strand DNA
9 third immobilized oligo DNA
10 still further extended DNA
11 47 mer 3′-beaded oligo DNA
12 48 mer 3′-beaded oligo DNA
13 T7 primer as starting DNA
14 SP6 primer for closing sequence
15 T7 primer
16 SP6 primer
17 67 nucleotide length of DNA
17a 5′ end of 67 mer DNA
17b 3′ end of 67 mer DNA
18 67 bp length
19 extended sample having 67 bp
20 marker for 67 mer DNA experiment
21 positive control for 67 mer DNA experiment
22 negative control for 67 mer DNA experiment
23-29 49 mer 3′-beaded oligo DNA
30 SP6 primer as starting DNA
31 T7 primer for closing sequence
32 241 bp length
33 extended sample having 241 bp (3-8-04 result)
33a 3-6-04 result for 241 mer DNA experiment
34 100 bp marker for 241 mer DNA experiment
35 10 bp marker for 241 mer DNA experiment
36 Negative control for 241 mer DNA experiment
37 annealing step
38 beads
39 extension step
40 denature step
41 second annealing step
42 second extension step
43 second denature step
44 spot 1
45 spot 2

What is claimed is:

1. A method of synthesizing a desired DNA having a predetermined sequence, characterized by the steps of:
(a) preparing, based on the desired DNA, a plurality of 3′-biotin-immobilized oligo DNA having a fixed length;
(b) preparing a starting DNA which has a complementary sequence to a 3′ end of a first immobilized oligo DNA of the plurality of oligo DNA, and wherein the starting DNA has a length shorter than the length of said first immobilized oligo DNA;
(c) annealing the starting DNA with said first immobilized oligo DNA, extending said starting DNA to complement said first immobilized oligo DNA, thereby making a newly extended DNA;
(d) denaturing a first double strand DNA consisting of said newly extended DNA and said first immobilized oligo DNA,
(e) collecting the extended DNA by removing the first immobilized oligo DNA from said extended DNA;
(f) annealing the collected, extended DNA with a second immobilized oligo DNA from the plurality of oligo DNA, further extending said extended DNA to complement said second oligo DNA, thereby making a further extended DNA;
(g) denaturing a second double stranded DNA consisting of said second immobilized oligo DNA and said further extended DNA;
(h) collecting the further extended DNA by removing said second immobilized oligo DNA from said extended DNA; and
(i) repeating steps (f) through (h) until said further extended DNA becomes the predetermined sequence thereby completing synthesis of the desired DNA.

2. The method of claim 1, wherein said oligo DNA is a 3-beaded oligo DNA, comprising a 3′-biotin modified DNA which is immobilized on beads.

3. The method of claim 1, wherein a 3′ end of said first immobilized oligo DNA matches at least 17 mer of a 5′ end of said starting DNA.

4. The method of claim 1, wherein step (e) and/or step (h) removes said—immobilized oligo DNA from said extended DNA by means of a magnet.

5. The method of claim 1, wherein step (e) and/or step (h) collects said extended DNA from said 3′-beaded oligo DNA and then reacts the next 3′-beaded oligo DNA by means of pipetting.

6. The method of claim 1, wherein step (e) and/or step (h) collects said extended DNA from said 3′-beaded oligo DNA and then reacts the next 3′-beaded oligo DNA by means of electrophoresis.

7. An apparatus for synthesizing a desired DNA having a predetermined sequence, which uses, characterized by:
(a) a first device for annealing a starting DNA with a first oligo DNA of a plurality of oligo DNA, where said starting DNA has a complementary sequence to a 3′ end of said first oligo DNA, and where said plurality of oligo DNA have a fixed length and said starting DNA has a length shorter than the length of said oligo DNA, thereby extending said starting DNA to complement said first oligo DNA and making a newly extended DNA;
(b) a second device for denaturing to remove said first oligo DNA from said extended DNA and collect said extended DNA;
(c) a third device for transferring the collected, extended DNA to a next oligo DNA from the plurality of oligo DNA;
(d) a fourth device for annealing said extended DNA with said next oligo DNA, further extending said extended oligo DNA to complement said next oligo DNA, thereby making a further extended DNA;
(e) a fifth device for denaturing to remove said next oligo DNA from said further extended DNA and collect said further extended DNA;
(f) a sixth device for transferring the collected, extended DNA to a next oligo DNA; and
(g) a seventh device for repeating steps (d) through (f) until said further extended DNA becomes the predetermined sequence thereby completing the desired DNA.

8. The apparatus of claim 7, wherein at least one of said devices are automated.

9. The apparatus of claim 7, wherein said oligo DNA is a 3-beaded oligo DNA (3′-biotin modified DNA).

10. The apparatus of claim 7, wherein a 3′ end of said first oligo DNA has a complementary sequence to at least 17mer of a 5′ end of said starting DNA.

11. The apparatus of claim 7, wherein in step (b) and/or step (e) said oligo DNA is removed from said extended DNA by means of a magnet.

12. The apparatus of claim 11, wherein said magnet means includes the dipping of the magnet into the solution containing said oligo DNA and said extended DNA.

13. The apparatus of claim 11, wherein said magnet means includes the placing of a magnet around the solution containing said oligo DNA and said extended DNA.

14. The apparatus of claim 7, wherein in step (b) and/or step (e) said extended DNA is removed from said oligo DNA by pipetting.

15. The apparatus of claim 7, wherein in step (b) and or step (e) said extended DNA is removed from said oligo DNA by electrophoresis.

16. The apparatus of claim 7, wherein in step (c) said removed extended DNA are transferred to a next reaction spot by pipetting guides.

17. The apparatus of claim 7, wherein in step (c) said removed extended DNA are transferred to a next reaction spot by electrophoresis guides.
